(19) United States
(12) Patent Application Publication
(10) Pub. No.: US 2010/0152244 A1
(43) Pub. Date: Jun. 17, 2010

(54) SELF-EMULSIFYING FORMULATION OF TIPRANAVIR FOR ORAL ADMINISTRATION

(75) Inventors: Sara Abelaira, Buenos Aires (AR); Mariela Paula Becher, Buenos Aires (AR); Juan Francisco Gel, Buenos Aires (AR); Maria Fernanda Villagra, Buenos Aires (AR); Marta Noemi Cicconi de Vidal, Buenos Aires (AR)

Correspondence Address: MICHAEL P. MORRIS;BOEHRINGER INGELHEIM USA CORPORATION, 900 RIDGEBURY ROAD, RIDGEFIELD, CT, 06877-0368, US

(73) Assignee: BOEHRINGER INGELHEIM INTERNATIONAL GMBH, Ingelheim am Rhein (DE)

(21) Appl. No.: 12/600,689

(22) PCT Filed: May 21, 2008
(86) PCT No.: PCT/EP08/56221

§ 371 Date: Nov. 18, 2009

(51) Int. Cl. A61K 31/4433; A61P 31/12

(52) U.S. Cl. 514/336

(57) ABSTRACT

A self-emulsifying formulation of tipranavir for oral administration.

### BACKGROUND OF THE INVENTION

1. Technical Field

The invention relates to a self-emulsifying formulation of tipranavir for oral administration.

2. Background Information

Tipranavir (also known as PNU 140690) is a non-peptidic HIV protease inhibitor which is useful for the treatment of HIV infection. Tipranavir has the following structural formula,

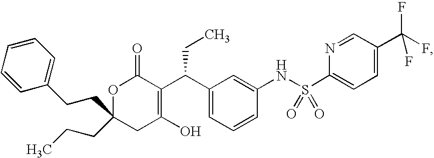

and is known by the following chemical names:
N-[3-[(1R)-1-[(6R)-5,6-dihydro-4-hydroxy-2-oxo-6-(2-phenylethyl)-6-propyl-2H-pyran-3-yl]propyl]phenyl]-5-(trifluoromethyl)-2-pyridinesulfonamide;
N-[3-[1-[5,6-dihydro-4-hydroxy-2-oxo-6-(2-phenylethyl)-6-propyl-2H-pyran-3-yl]propyl]phenyl]-5-(trifluoromethyl)-, [R-(R*,R*)]-2-pyridinesulfonamide; and,
3′-[(1R)-1-[(6R)-5,6-Dihydro-4-hydroxy-2-oxo-6-phenylethyl-6-propyl-2H-pyran-3yl]propyl]-5-(trifluoromethyl)-2-pyridinesulfonanilide.

The synthesis of tipranavir and the manner in which it may be used to treat HIV infection are described in U.S. Pat. No. 5,852,195 and published International Application WO9530670.

Several pharmaceutical formulations of tipranavir have been described in the literature, as summarized below.

U.S. Pat. No. 6,531,139 and the corresponding published International Application WO9906024 describe a pharmaceutical composition which comprises a lipophilic, pharmaceutically active agent (specifically including but not limited to tipranavir), a lipid which is a mixture of mono- and diglycerides, a solvent and a surfactant. A number of pharmaceutically acceptable solvents are listed, including polyethylene glycol, although propylene glycol is stated to be the preferred solvent. A number of pharmaceutically acceptable surfactants are listed, with Cremophor RH40® or Cremophor EL® being preferred. Vitamin E TPGS is not included in the listing of pharmaceutically acceptable surfactants. These cited references indicate that the composition described therein, which is a liquid, may be used to fill capsules for oral administration, and that it may also be in the form of a liquid solution for oral, parenteral, rectal or topical application.

The disclosures of U.S. Pat. No. 6,121,313 and the corresponding published International Application WO9906043 are essentially the same as that of U.S. Pat. No. 6,531,139 and the corresponding published International Application WO9906024 described above, but the pharmaceutically active agent is limited to certain pyranones, specifically including but not limited to tipranavir.

U.S. Pat. No. 6,231,887 and the corresponding published International Application WO9906044 describe a pharmaceutical composition which comprises a pyranone (specifically including but not limited to tipranavir) as a pharmaceutically active agent, a basic amine, a solvent and a surfactant, and optionally a lipid which is a mixture of mono- and diglycerides. A number of pharmaceutically acceptable solvents are listed, including polyethylene glycol, although propylene glycol is stated to be the preferred solvent. A number of pharmaceutically acceptable surfactants are listed, with Cremophor RH40® or Cremophor EL® being preferred. Vitamin E TPGS is not included in the listing of pharmaceutically acceptable surfactants. It is indicated that the composition thus described, which is a liquid, may be used to fill capsules for oral administration, and that it may also be in the form of a liquid solution for oral, parenteral, rectal or topical application.

U.S. Pat. No. 6,555,558 and the corresponding published International Application WO0236110 describe a pharmaceutical composition which comprises a pyranone protease inhibitor (specifically including but not limited to tipranavir), a surfactant, a polyethylene glycol solvent, a lipid which is a mixture of mono- and diglycerides and, optionally, a basic amine. The composition is substantially free of ethanol and propylene glycol. A number of pharmaceutically acceptable surfactants are listed, with Cremophor EL® being preferred. Vitamin E TPGS is not included in the listing of pharmaceutically acceptable surfactants. It is indicated that the composition thus described, which is a liquid, is particularly suitable for filling soft gelatin capsules intended for oral administration.

Vitamin E-TPGS (d-Alpha Tocopheryl Polyethylene Glycol 1000 Succinate) is a water soluble form of vitamin E and has been recognized as an excipient to promote emulsification of lipophilic substances, acting as a non-ionic surfactant, and in improving the bioavailability of certain drugs.

For example, in The Lancet, 1991. 338, 212-214 Sokol R. J. et al teaches that coadministration of Vitamin E-TPGS with cyclosporin improves the bioavailability of cyclosporin.

U.S. Pat. No. 6,193,985 and the corresponding published International Application WO9531217 describe the use of tocopherols as solvents and/or emulsifiers of drugs that are substantially insoluble in water, in particular for the preparation of topical formulations. Use of Vitamin E-TPGS is specifically mentioned at pages 7-8 and 12 as an emulsifier for use in formulations containing high levels of alpha.-tocopherol as the lipid layer. Examples of formulations for topical administration disclosed containing Vitamin E-TPGS, such as Examples 1 to 5, typically comprises a lipid layer (an .alpha.-tocopherol), the drug and Vitamin E-TPGS, in quantities of less than 25% w/w of the formulation, as an emulsifier. This reference does not describe any formulation of an HIV protease inhibitor.

WO96/36316 teaches that Vitamin E-TPGS can be used for the enhanced delivery of lipophilic compounds as a self-emulsifying preconcentrate formulation comprising a) a lipophilic drug (a cyclosporin is specifically exemplified), b) vitamin E-TPGS and c) a lipophilic phase. Typical examples of formulations disclosed, such as Examples 2 and 4, contain less than 14% w/w Vitamin E-TPGS as an emulsifier, a lipid layer and the drug. There is no reference to formulation of HIV protease inhibitors.

Finally, U.S. Pat. No. 6,730,679, the corresponding published International Application WO9735587 and Yu et al., Pharm Res. 1999 December; 16(12):1812-7 describe pharmaceutical compositions containing amprenavir, an HIV protease inhibitor, and Vitamin E-TPGS.

It is believed that there are not yet any formulations of tipranavir which are particularly well adapted for oral administration in the form of an unencapsulated liquid. Such a formulation would be particularly suitable for pediatric patients and also for adults who have difficulty swallowing solids.

Thus, it is the object of the present invention to provide such a liquid formulation of tipranavir.

### BRIEF SUMMARY OF THE INVENTION

The present invention provides a pharmaceutically acceptable, self-emulsifying oral formulation of tipranavir in the form of a solution for oral administration.

Based upon a generally accepted belief that a self-emulsifying drug delivery system should contain at least one lipid excipient as the lipid phase (in order to achieve effective emulsification upon dilution in the GI tract and thereby the improved bioavailability for the drug), we first attempted to develop an oral solution of tipranavir which included Capmul®MCM as a lipid phase. Capmul® MCM is a mono-diglyceride of medium chain fatty acids (mainly caprylic and capric) and its use as a lipid emulsifier is quite conventional in the pharmaceutical art. The composition of this formulation is shown in Table 1.

TABLE I
Tipranavir Oral Solution Formulation (F173)

| Ingredient | mg/mL | Function |
|---|---|---|
| Tipranavir | 100.0 | Drug Substance |
| Polyethylene Glycol 400 | 417.0 | Solvent |
| Propylene Glycol | 80.0 | Solvent |
| Capmul ® MCM | 30.0 | Lipid Phase |
| (Mono/Diglycerides of | | |
| Caprylic/Capric Acid) | | |
| Vitamin E Polyethylene Glycol | 300.0 | Surfactant |
| Succinate | | |
| Ascorbic Acid | 2.0 | Anti-oxidant |
| Water, Purified | 150.0 | Solvent |
| Sucralose | 20.0 | Sweetening agent |
| Buttermint 24020 | 10.0 | Flavor |
| Butter Toffee 78185-33 | 10.0 | Flavor |
| Total Weight | 1119.0 | |

The formulation described in Table I was tested in clinical studies and shown to provide adequate in vivo bioavailability. However, this formulation was, quite unexpectedly, found physically unstable (due to precipitation) after long term storage at room temperature.

At first the nature of the precipitate and its cause were unknown. However, after extensive investigation, we discovered that the precipitation was due to the completely unexpected formation of a new solid form, a co-crystal in the oral solution. This co-crystal form consists of tipranavir and 1,3-dioctanylglycerol (1,3-DOG) non-covalently bounded at a 4-to-1 molar ratio. The 1,3-DOG is a lipid component from Capmul MCM. The cocrystal has a lower solubility than Tipranavir and therefore precipitates out from the solution. The chemical structure of the cocrystal is shown in FIG. 1.

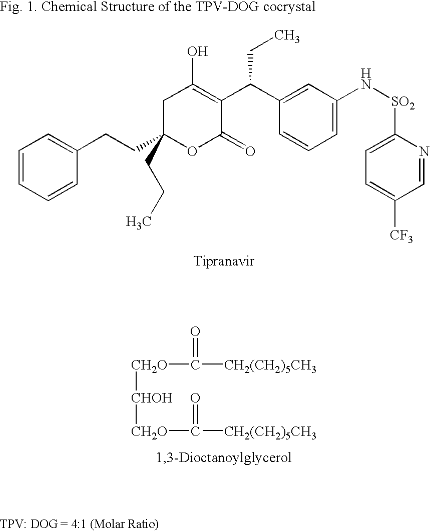

Once the cause of the problem was understood a counter-intuitive solution was devised. We discovered that the lipid phase (Capmul® MCM) could be omitted from the formulation and that, contrary to conventional wisdom, the lipid-free formulation functioned nicely. The lipid-free formulation exhibits in vitro self-emulsifying properties that are similar to the Capmul-containing formulation (FIG. 2).

One possible reason for the self emulsifying behavior of the Capmul-free formulation is that tipranavir which is a highly lipophilic compound (Log P=6) once dissolved serves as a lipid core in this pre-concentrated microemulsion system. However, this rationale is only a theory and it was certainly not available to serve as guidance and motivation to us when we set about to solve the instability problem posed by the Capmul-containing formulation. This new formulation is expected to show similar in vivo performance based on the in vitro and in vivo correlation in dogs. The new Capmul MCM-free formulation of the invention has no precipitation issue and is physically and chemically stable.

### DETAILED DESCRIPTION OF THE INVENTION

The self-emulsifying, liquid formulation of the invention comprises:

(a) tipranavir,
(b) Vitamin E TPGS as a surfactant;
(c) one or more pharmaceutically acceptable solvents
The formulation does not require a lipid phase.

The active ingredient, tipranavir, is present in an amount from 1% to 40% by weight of the total composition.

Vitamin E TPGS comprises 10% to 50% by weight of the total composition.

Pharmaceutically acceptable solvents suitable for use in the context of the present invention are propylene glycol, polypropylene glycol, polyethylene glycol (such as PEG300, 400, 600, etc.), glycerol, ethanol, triacetin, dimethyl isosorbide, glycofurol, propylene carbonate, water, dimethyl acetamide or a mixture thereof. The preferred solvent is a mixture of water, polyethylene glycol having a mean molecular weight of greater than 300 but lower than 600 and propylene glycol. Still more preferred as solvent is a mixture of water, propylene glycol and polyethylene glycol 400. The solvent, or mixture of solvents, comprises 10% to 60% by weight of the total composition.

The formulation in accordance with the invention optionally includes further excipients and/or flavoring agents. Thus, for example, it is preferred to include an anti-oxidant such as ascorbic acid, and agents to sweeten or flavor the formulation. Those of ordinary skill in the pharmaceutical art will know how to select acceptable anti-oxidant, sweetening or flavoring agents.

The term “self-emulsifying formulation” used herein refers to a concentrated composition capable of generating emulsions or microemulsions upon mixing with sufficient aqueous media.

Thus, the self-emulsifying formulations in accordance with the present invention generate emulsions or microemulsions when mixed with aqueous media. The formulation can be mixed with an aqueous medium such as water, fruit juice or the like, prior to ingestion, and the resulting emulsion can then be ingested. Alternatively, the formulation can be ingested in either liquid or encapsulated form so that it will mix with gastric fluid, forming an emulsion in situ.

The emulsions or microemulsions generated from the present invention are solutions comprising a hydrophilic phase and a lipophilic phase (in this case tipranvir). Microemulsions are also characterized by their thermodynamic stability, optical transparency and small average droplet size, generally less than about 0.15 micron.

The amount of the active ingredient tipranavir in the composition may vary or be adjusted widely depending on the intended route of administration, the potency of the particular active ingredient being used, the severity of the retroviral infection and the required concentration.

The invention is further illustrated by means of the following working example.

### Example 1

### Self-Emulsifying Formulation of Tipranavir

The following ingredients in Table 2 were mixed to form a liquid formulation.

TABLE 2
Tipranavir Oral Solution Formulation (F347)

| Ingredient | mg/mL | Function |
|---|---|---|
| Tipranavir | 100.0 | Drug Substance |
| Polyethylene Glycol 400 | 457.0 | Solvent |
| Propylene Glycol | 80.0 | Solvent |
| Vitamin E Polyethylene Glycol | 290.0 | Surfactant |
| Succinate | | |
| Ascorbic Acid | 2.0 | Anti-oxidant |
| Water, Purified | 150.0 | Solvent |
| Sucralose | 20.0 | Sweetening agent |
| Buttermint 24020 | 10.0 | Flavor |
| Butter Toffee 78185-33 | 10.0 | Flavor |
| Total Weight | 1119.0 | |

Based on an established in vitro and in vivo correlation in dogs, this formulation is expected to show similar bioavailability as the lipid-containing formulation (F173).

What is claimed is:

1. A self-emulsifying, liquid pharmaceutical composition which comprises:
(a) tipranavir,
(b) Vitamin E TPGS as a surfactant; and
(c) one or more pharmaceutically acceptable solvents.

2. A pharmaceutical composition in accordance with claim 1, wherein the pharmaceutically acceptable solvent is propylene glycol, polypropylene glycol, polyethylene glycol (such as PEG300, 400, 600, etc.), glycerol, ethanol, triacetin, dimethyl isosorbide, glycofurol, propylene carbonate, water, dimethyl acetamide or a mixture thereof.

3. A pharmaceutical composition in accordance with claim 2, wherein the solvent is a mixture of water, polyethylene glycol having a mean molecular weight of greater than 300 but lower than 600 and propylene glycol.

4. A pharmaceutical composition in accordance with claim 3, wherein the solvent is a mixture of water, propylene glycol and polyethylene glycol 400.

5. A pharmaceutical composition in accordance with claim 1 wherein the solvent, or mixture of solvents, comprises 10% to 60% by weight of the total composition.

6. A pharmaceutical composition in accordance with claim 1 wherein Vitamin E TPGS comprises 10% to 50% by weight of the total composition.

7. A pharmaceutical composition comprising:

| | | |
|---|---|---|
| Tipranavir | 100.0 | mg/mL, |
| Polyethylene Glycol 400 | 457.0 | mg/mL, |
| Propylene Glycol | 80.0 | mg/mL, |
| Vitamin E Polyethylene Glycol | 290.0 | mg/mL, |
| Succinate | | |
| Ascorbic Acid | 2.0 | mg/mL, |
| Water, Purified | 150.0 | mg/mL, |
| Sucralose | 20.0 | mg/mL, |
| Buttermint 24020 | 10.0 | mg/mL, and |
| Butter Toffee 78185-33 | 10.0 | mg/mL. |
